(19) United States
(12) Patent Application Publication
(10) Pub. No.: US 2024/0408369 A1
(43) Pub. Date: Dec. 12, 2024

(54) ACCESS DEVICE

(71) Applicant: THE GENERAL HOSPITAL CORPORATION, Boston, MA (US)

(72) Inventors: Graham M.F. Lieberman, Boston, MA (US); Hilary Y. Gallin, Boston, MA (US)

(21) Appl. No.: 18/699,271

(22) PCT Filed: Oct. 5, 2022
(86) PCT No.: PCT/US2022/045764

§ 371 Date: Apr. 6, 2024

Related U.S. Application Data
(60) Provisional application No. 63/253,300, filed on Oct. 7, 2021.

(51) Int. Cl. A61M 39/02; A61B 90/00

(52) U.S. Cl. CPC A61M 39/02; A61B 90/06; A61B 2090/064

(57) ABSTRACT

An access device includes an access assembly for percutaneously accessing an anatomical space in a patient. An aspiration assembly is operable to generate negative pressure in a fluidic channel of the device. The fluidic channel places the access assembly in fluid connection with the aspiration assembly. A fluid tube has a first tube end connected to the access assembly, a second tube end connected to the aspiration assembly, and a tube body. The tube body defines a manometer tube portion spaced apart from both the first and second tube ends. The manometer tube portion acts as at least part of a manometer when the device is in a manometer mode. The manometer tube portion has adjacent first and second manometer tube ends and an inflection point substantially reversing the direction of the manometer tube portion at a location spaced apart from both the first and second manometer tube ends.

### RELATED APPLICATION

This application claims priority from U.S. Provisional Application No. 63/253,300, filed 7 Oct. 2021, the subject matter of which is incorporated herein by reference in its entirety for all purposes.

### TECHNICAL FIELD

This disclosure relates to an access device and a method for accessing an anatomical space in a patient.

### BACKGROUND

The Seldinger technique is a common maneuver in the field of medicine used to insert a cannula into a vessel, hollow organ, or body cavity. Examples of procedures and settings where it may be employed include arterial line placements, central venous catheter placements, angiograms, percutaneous tracheostomies, pleural catheter placement, percutaneous cholecystectomies, percutaneous nephrostomies, and percutaneous abscess drainage. With respect to vascular access, it is the current standard of care for placing a catheter, sheath, or cannula into a blood vessel during access procedures such as central venous catheter placement. In applying the Seldinger technique, a physician first uses a needle to pierce through tissue to ultimately reach a targeted body space. A wire is then passed through the needle into the space. The wire secures a path into the space over which the needle may then be removed and additional instruments such as sheaths and catheters may be inserted into the space.

With respect to central venous catheter insertion and procedures targeting lumens with similarly low-pressure fluids like blood in the venous system, a syringe is coupled to the needle to apply suction. Once the physician gains access to the targeted body space, which is confirmed when a predetermined body fluid is aspirated into the syringe barrel, the physician holds the hub of the needle with one hand and uncouples the syringe with from the needle with the remaining hand. Once the uncoupling is complete, the physician inserts a guidewire through the lumen of the needle into the targeted body space in order to secure access. The physician then removes the needle, nicks the skin with a scalpel, and passes a dilator over the guidewire to dilate the tissue around the guidewire in order to facilitate catheter, sheath, or cannula insertion. Once the dilator is withdrawn, the physician passes the catheter, sheath, or cannula over the guidewire, leaving the catheter, sheath, or cannula in place.

The use of ultrasound guidance during placement of a catheter, sheath, or cannula is well-documented. The precise identification of vessel anatomy and visualization of the puncture site that is enabled through the use of ultrasound may be helpful in vascular access. Ultrasound guidance also allows the clinician to visually locate the needle tip relative to patient anatomy in real time and further confirm wire, catheter, sheath, or cannula placement thereafter.

The ultrasound-guided Seldinger technique is implemented in a manner largely similar to the Seldinger technique in many respects. During the needle insertion step, the physician uses the syringe with a needle in one hand-typically the operator's dominant hand—and an ultrasound probe in the other hand in order to visualize the needle's trajectory and location inside the patient's body using alternating longitudinal and cross-sectional views with the ultrasound probe. As with the Seldinger technique, once the operator gains access to the targeted body space, which is confirmed when a specific body fluid is aspirated into the syringe's barrel, the operator holds the needle's hub with one hand and uncouples the syringe from the needle with the remaining hand. During this step, the ultrasound probe is temporarily set aside to permit the uncoupling, resulting in loss of ultrasound visualization of the needle in the targeted space. The removal of the ultrasound probe from the patient also releases the pressure of the probe on the tissue, which can result in unwanted movement of tissue relative to the needle tip.

Because the ultrasound-guided Seldinger technique (and the standard Seldinger technique) utilizes both of the physician's hands to uncouple the needle from the syringe, stabilize the needle, and insert the guidewire into the targeted body space after the initial needle insertion step, there is a period of time during the procedure in which a sharp needle is located inside of a patient's body while the physician or other clinician cannot visualize it and has limited control over it. In particular, once the needle tip is inside the targeted body space, the clinician drops the ultrasound probe and performs hand switching motions that can undesirably move the needle. The clinician also uncouples the syringe from the needle in a step that can also move the needle and reduce needle control. Further, the clinician passes a guidewire through the needle's lumen in a process that can also move the needle relative to the targeted body space. The clinician passes the guidewire without seeing the wire trajectory and location of the wire tip in real time. While ultrasound guidance helps with initial needle location and access, its advantages may be lost during the multiple steps requiring the use of both of an operator's hands for non-ultrasound tasks. Ultrasound guidance may be reinstated for verification after these steps, but at this point needle access may already be lost. The wire could also be threaded into other, non-target tissues.

### SUMMARY

The invention described herein has been designed at least partially with the above issues/complications in mind. Further, the invention described herein may similarly be applied in settings where the Seldinger technique is used, especially but not exclusively with ultrasound.

In an aspect, alone or in combination with any other aspect, an access device is provided. The device includes an access assembly for percutaneously accessing an anatomic space in a patient. An aspiration assembly is selectively operable to generate negative pressure in a fluidic channel of the device. The fluidic channel places the access assembly in fluid connection with the aspiration assembly. A fluid tube has a first tube end connected to the access assembly, a second tube end connected to the aspiration assembly, and a tube body extending between the first and second tube ends. The tube has a tube lumen that extends between the first and second tube ends and at least partially defines the fluidic channel between the access assembly and the aspiration assembly. The tube body defines a manometer tube portion spaced apart from both the first and second tube ends. The manometer tube portion selectively forms a manometer when the device is selectively in a manometer mode. The manometer tube portion has adjacent first and second manometer tube ends and an inflection point substantially reversing the direction of the manometer tube portion at a location spaced apart from both the first and second manometer tube ends.

In an aspect, alone or in combination with any other aspect, a method for accessing an anatomical space in a patient is provided. The method includes providing an access device. A portion of an access assembly of the device is inserted into the patient. An aspiration assembly of the device is operated to generate the negative pressure. Via the negative pressure, fluid from the patient is aspirated into the manometer tube portion. The device is manipulated into a manometer operating mode such that a manometer tube portion of a fluid tube of the device forms a manometer. With the device in the manometer operating mode, the fluid within the manometer tube portion is monitored to responsively determine whether the inserted portion of the access assembly is in a target anatomical space within the patient.

### BRIEF DESCRIPTION OF THE DRAWINGS

For a better understanding, reference may be made to the accompanying drawings, in which:

FIG. 1 is a perspective side view of an access device according to one aspect of the present invention, including the device in a first condition;

FIG. 2 is a perspective side view of a portion of the device of FIG. 1;

FIG. 3 is a perspective bottom view of a portion of the device of FIG. 1;

FIG. 4 schematically illustrates a portion of a component of the device of FIG. 1;

FIG. 5 is a side view of a component of the device of FIG. 1;

FIG. 6 is a perspective side view of the device of FIG. 1 in a second condition; and

FIGS. 7-16 schematically illustrate an example sequence of operation for accessing an anatomical space in a patient.

### DESCRIPTION OF EMBODIMENTS

Unless defined otherwise, all technical and scientific terms used herein have the same meaning as is commonly understood by one of ordinary skill in the art to which the present disclosure pertains.

As used herein, the term “patient” can refer to any warm-blooded organism including, but not limited to, human beings, pigs, rats, mice, birds, cats, dogs, goats, sheep, horses, monkeys, apes, rabbits, cattle, farm animals, livestock, etc.

As used herein, the term “user” can be used interchangeably to refer to an individual who prepares for, assists with, and/or performs a procedure, and/or an individual who prepares for, assists with, and/or performs the operation of a tool.

As used herein, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

As used herein, phrases such as “between X and Y” can be interpreted to include X and Y.

As used herein, the phrase “at least one of X and Y” can be interpreted to include X, Y, or a combination of X and Y. For example, if an element is described as having at least one of X and Y, the element may, at a particular time, include X, Y, or a combination of X and Y, the selection of which could vary from time to time. In contrast, the phrase “at least one of X” can be interpreted to include one or more Xs.

It will be understood that when an element is referred to as being “on,” “attached” to, “connected” to, “coupled” with, etc., another element, it can be directly on, attached to, connected to or coupled with the other element or intervening elements may also be present. It will also be appreciated by those of skill in the art that references to a structure or feature that is disposed “adjacent” another feature may not have portions that overlap or underlie the adjacent feature.

Spatially relative terms, such as “below,” “above” and the like, may be used herein for ease of description to describe one element or feature's relationship to another element(s) or feature(s) as illustrated in the Figures. It will be understood that the spatially relative terms can encompass different orientations of a device in use or operation, in addition to the orientation depicted in the Figures. For example, if a device in the Figures is inverted, elements described as “below” other elements or features would then be oriented “above” the other elements or features.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or Figures unless specifically indicated otherwise.

The invention comprises, consists of, or consists essentially of the following features, in any combination.

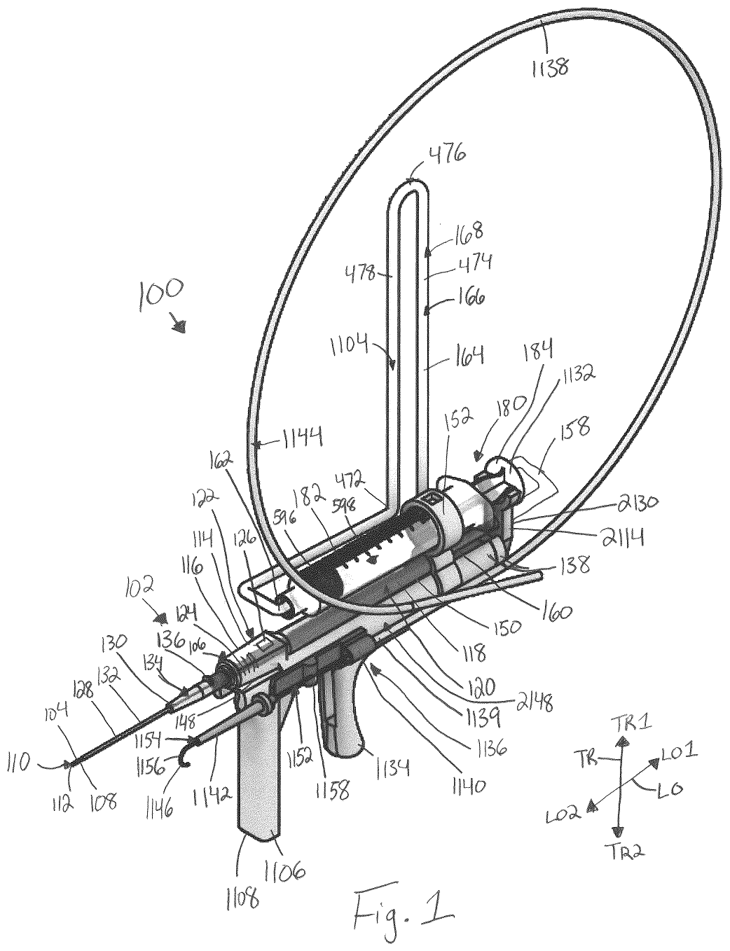

FIG. 1 depicts an access device 100, such as a percutaneous vascular access device 100. The device 100 includes an access assembly 102 for percutaneously accessing, any anatomic space in a patient, such as, for example any patient lumen, organ, and/or body cavity. For the sake of discussion, a vasculature (e.g., a vein and/or an artery) of a patient will be used as an example use environment herein. The access assembly 102 includes a hollow needle 104 having longitudinally opposite proximal and distal needle ends 106, 108, as well as a needle lumen 110 extending longitudinally between the proximal and distal needle ends 106, 108. The term “longitudinal” is used herein to describe a direction substantially parallel to the needle lumen, with the “longitudinal” direction being indicated as “LO” in FIG. 1. The distal needle end 108 has a sharp distal needle tip 112 for suitable for piercing the patient's skin. The proximal needle end 106 is connected to an access housing 114.

The access housing 114 has an access housing head portion 116 and an access housing body portion 118 that is longitudinally proximal (as shown by an arrow “LO1” in FIG. 1) to the access housing head portion 116. At least one of the access housing body portion 118 and the access housing head portion 116 defines or includes an inner access housing lumen 120 that extends longitudinally through at least a portion of the access housing 114 and is fluidically connected to the needle lumen 110.

At least the access housing head portion 116 may include a needle retraction mechanism 122 therein. However, at least a portion of the needle retraction mechanism 122 may also or instead be in the access housing body portion 118. The needle retraction mechanism 122 is configured to selectively move the needle 104 between deployed and retracted positions. In the deployed position, the needle 104 extends longitudinally distally (as shown by an arrow “LO2” in FIG. 1) from the access housing head portion 116 so that the distal needle tip 112 is outside the access housing 114 and in position for selectively piercing the patient's skin. In the retracted position, an entirety of the needle 104 (including the distal needle tip 112) is in the access housing 114 (e.g., in the access housing lumen 120) so as to help prevent accidental needle sticks.

The needle retraction mechanism 122 may include any components suitable for longitudinally moving the needle 104 between the deployed and retracted positions. The needle retraction mechanism 122, for example, may include at least a spring 124 and a retraction trigger 126. The spring 124 may be configured to bias the needle 104 toward the retracted position. The spring 124 may be compressed against the spring bias as the needle 104 is moved to the deployed position. Once the needle 104 is in the deployed position, the spring 124 may be selectively held in a compressed state by a portion of the retraction trigger 126 (or an intermediate component joined to the retraction trigger 126) to retain the needle 104 in that deployed position. When retraction of the needle 104 is desired, a user may actuate (such as by depressing) the retraction trigger 126 so that the retraction trigger 126 may disengage the spring 124 and permit the spring 124 to expand from the compressed state. As the spring decompresses, the spring 124 urges the needle 104 to the retracted position inside the access housing 114.

The access assembly 102 also includes a catheter 128. The catheter 128 includes a catheter hub 130 that removably couples the catheter 128 to the access housing head portion 116. A catheter body 132 is connected to and extends longitudinally distally from the catheter hub 130. The catheter hub and body 130, 132 define a catheter lumen 134 that extends longitudinally through the catheter 128. As shown in FIG. 1, at least a portion of the needle 104 may extend through the catheter lumen 134 when the needle 104 is in the deployed position. The catheter 128 and the needle 104 are configured such that the catheter 128, when uncoupled from the accessing housing head portion 116, may be slid longitudinally along the deployed needle 104 as desired. The catheter hub 130 may include a catheter projection 136 extending outwardly therefrom that the user may act against when uncoupling and sliding the catheter 128.

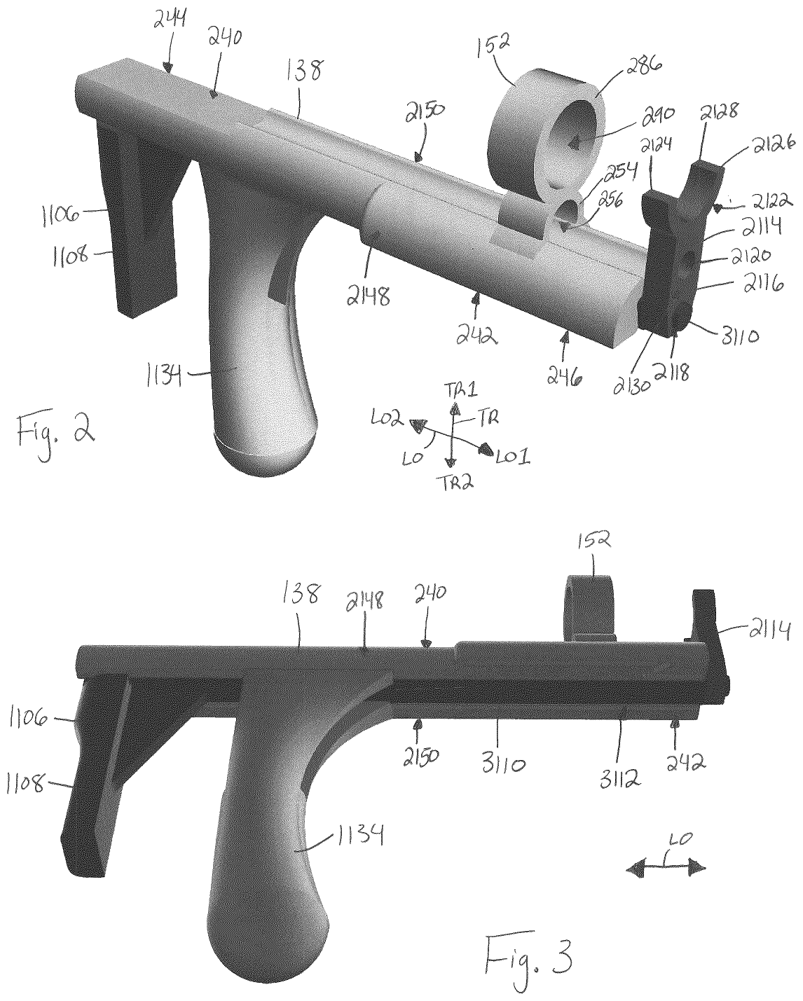

At least a portion of the access housing 114 is positioned on a barrel 138 of the device 100. As shown in FIGS. 2-3, the barrel 138 includes transversely opposite and spaced apart first and second barrel surfaces 240, 242. The term “transverse” is used herein to indicate a direction substantially perpendicular to the “longitudinal” direction, and the “transverse” direction is substantially vertical in the orientation of FIG. 2, as well as being indicated at “TR” in FIGS. 1-2. As shown in FIGS. 1-2, the first barrel surface 240 at a distal barrel portion 244 may be configured to receive the access housing head portion 116 thereon, while the first barrel surface 240 at a proximal barrel portion 246 may be configured to receive the access housing body portion 118 thereon. In the configuration shown in FIGS. 1-2, the first barrel surface 240 at the distal barrel portion 244 may be substantially flat (i.e., free from curves or projections) for receiving a correspondingly flat head engagement surface 148 of the access housing head portion 116. The first barrel surface 240 at the proximal barrel portion 246 may instead be at least partially curved, rounded, or otherwise contoured for receiving a correspondingly shaped body engagement surface 150 of the access housing body portion 118.

The access housing 114 (and, accordingly, the access assembly 102 as a whole) may be removably held in the above discussed position on the barrel 138 via a barrel connector 152. As shown in FIGS. 1-2, the barrel connector 152 extends transversely from the first barrel surface 240. The barrel connector 152 may be formed integrally with the barrel 138 as a one-piece, unitary component, or separately from the barrel and subsequently permanently or removably connected to the barrel 138. A first portion 254 of the barrel connector 152 and the rounded or curved portion of the first barrel surface 240 may collectively define an access housing opening 256 configured to receive the access housing body portion 118, though the access housing opening 256 can be configured to additionally or instead receive the access housing head portion 116. When received in the access housing opening 256, the access housing body portion 118 may be retained in the access housing opening 256 and in the desired position on the first barrel surface 240 via an interference or frictional fit between the access housing body portion 118 and the portions of the barrel connector 152 and the first barrel surface 240 that define the access housing opening 256.

Although the access housing 114 has been described as being connected to and maintained connected to the barrel 138 via the interference or frictional fit, any other suitable engagement method may be provided for the same purpose, such as, but not limited to, a magnetic engagement, hook and loop engagement, a nail and nail hole engagement, a screw and screw hole engagement, a snap-fit engagement, an adhesive engagement, a screw and nut engagement, a split pin engagement, any other suitable engagement, or any combination thereof. Additionally, because the access assembly 102 is removably coupled to the barrel 138, a user may remove and replace the access assembly 102 with a different access assembly 102 or another component/assembly as desired. Although the access assembly 102 has been described as being removably connected to the barrel 138, a portion of the access assembly 102 (e.g., the access housing head portion 116 and/or the access housing body portion 118) may be permanently connected to the barrel 138. When permanently connected, the connected portion(s) of the access assembly 102 may be formed integrally with the barrel 138 as a one-piece, unitary component, or separately from the barrel 138 and subsequently permanently connected to the barrel 138.

As shown in FIG. 1, the device includes a fluid tube 158 having a first tube end 160, a second tube end 162, and a tube body 164 extending between the first and second tube ends 160, 162. The fluid tube 158 also has a tube lumen 166 that extends between the first and second tube ends 160, 162. The first tube end 160 is connected (removably or permanently) to the access housing body portion 118 such that the access housing lumen 120 is fluidically connected to the tube lumen 166.

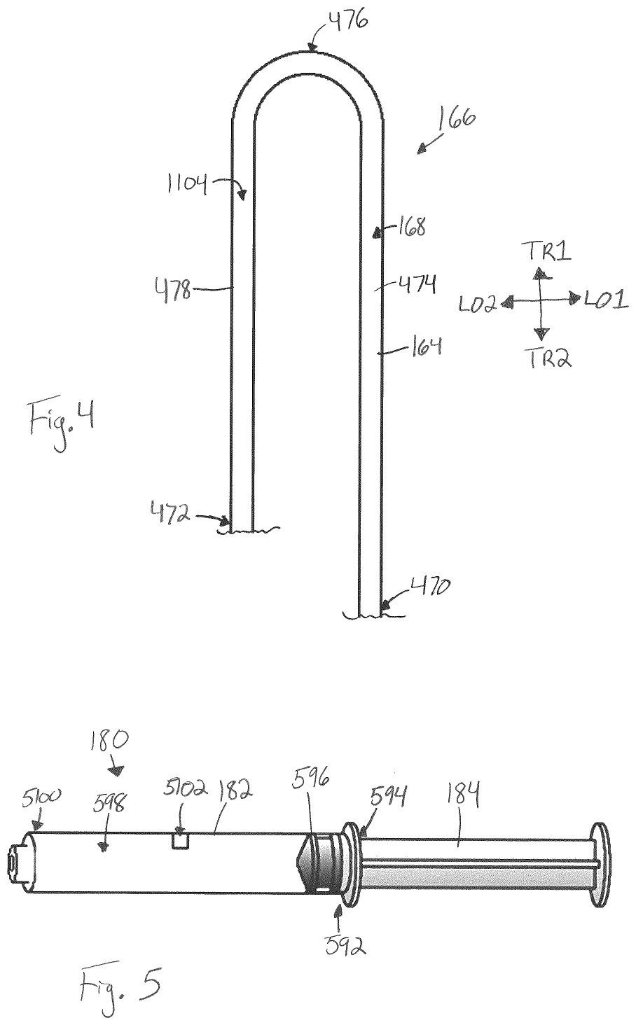

The tube body 164 defines a manometer tube portion 168 spaced apart from both the first and second tube ends 160, 162. As will be discussed in more detail below, the manometer tube portion 168 acts as at least part of a manometer when the device 100 is selectively in a manometer mode. As shown in FIG. 4, the manometer tube portion 168 has adjacent first and second manometer tube ends 470, 472. Each of the first and second manometer tube ends 470, 472 may open in the same (transverse) direction. A first manometer tube leg 474 extends in a first transverse direction (shown as an arrow “TR1” in FIG. 4) from the first manometer tube end 470 to an inflection point 476, while a second manometer tube leg 478 extends in a opposite, second transverse direction (shown as an arrow “TR2” in FIG. 4) from the inflection point 476 to the second manometer tube end 472. The term “inflection point” is used herein to indicate an area of the manometer tube portion 168 wherein a change of extension direction of at least 45° occurs; an inflection “point” need not be a sudden change of direction, but could be a gradual change over a predetermined area of the manometer tube portion 168. Therefore, as shown in FIG. 1, the first manometer tube leg 474 extends transversely away from the barrel 138, and the second manometer tube leg 478 extends transversely toward the barrel 138. The inflection point 476 thus denotes a substantial reversal of the extension direction of the manometer tube portion 168 at a location which is spaced apart from both the first and second manometer tube ends 470, 472. As shown in at least FIG. 4, the first manometer tube leg 474 may extend further in the transverse direction TR than does the second manometer tube leg 478. As such, the first manometer tube end 470 may be spaced transversely closer to the barrel 138 than does the second manometer tube end 472.

The inflection point 476, as is shown in FIGS. 1 and 4, is rounded and helps shape the manometer tube portion 168 in the form of an inverted “U”. Therefore, in the configuration shown in FIGS. 1 and 4, the manometer tube portion 168 (i.e., first and second manometer tube ends 470, 472, the first and second manometer tube legs 474, 478, and the inflection point 476) collectively forms an inverted U-shaped manometer when the device 100 is selectively in the manometer mode. However, instead of being rounded, the inflection point 476 may instead have a sharp point such that the manometer tube portion 168 is shaped as an inverted “V”, may be more “squared-off” with two “corners,” or may have any other desired form. Further, although the manometer tube portion 168 is shown as generally extending in the first transverse direction TR1 away from the barrel 138 such that the inflection point 476 is transversely “above” the barrel 138 in the orientation of FIG. 1, the manometer tube portion 168 may generally extend in the second transverse direction TR2 away from the barrel 138 such that the inflection point 476 is transversely “below” the barrel 138 in the orientation of FIG. 1. In the “transversely below” configuration, the manometer tube portion 168 may selectively form a U-shaped manometer (not an inverted U-shaped manometer) when the device 100 is selectively in the manometer mode.

As shown in FIG. 1, the second tube end 162 is connected to an aspiration assembly 180. As shown in FIGS. 1 and 5, the aspiration assembly 180 includes an aspiration body 182 and a plunger 184. The aspiration body may be removably connected to the barrel 138 (e.g., to the first barrel surface 240) via the barrel connector 152. In particular, as shown in FIGS. 1-2, a second portion 286 of the barrel connector 152 extends transversely from the first portion 254 away from the barrel 138. The second portion 286 of the barrel connector 152 includes an aspiration body opening 290 configured to receive the aspiration body 182. When received in the aspiration body opening 290, the aspiration body 182 may be retained in the aspiration body opening 290 and connected to the barrel 138 via an interference or frictional fit between the aspiration body 182 and the second portion 286 of the barrel connector 152. As shown in FIG. 1, the connected aspiration body 182 may be positioned transversely above (in the orientation of FIG. 1) at least a portion of the access assembly 102 and may extend substantially longitudinally in parallel with the access assembly 102, though the aspiration assembly 180, the access assembly 102, and the barrel 138 may be arranged in any other desired manner.

Although the aspiration body 182 has been described as being connected to and maintained in connection with the barrel 138 via an interference or frictional fit, any other suitable engagement method (e.g., an adhesive, a coupler, a penetrating fastener, and/or any other desired type) may be provided for the same purpose. Additionally, because the aspiration assembly 180 is removably coupled to the barrel 138, a user may remove and replace the aspiration assembly 180 with a different aspiration assembly 180 or another component/assembly as desired. Although the aspiration assembly 180 has been described as being removably connected to the barrel 138, a portion of the aspiration assembly 180 (e.g., the aspiration body 182) may be permanently connected to the barrel 138. When permanently connected, the connected portion of the aspiration assembly 180 may be formed integrally with the barrel 138 as a one-piece, unitary component, or separately from the barrel 138 and subsequently permanently connected to the barrel 138.

As shown in FIG. 5, the aspiration body 182 includes a proximal aspiration end 592 having a proximal aspiration opening 594 through which at least a plunger head 596 of the plunger 184 may be inserted (e.g., removably inserted) longitudinally into the aspiration body 182. When the plunger head 596 is inserted into the aspiration body 182, the plunger head 596 forms a circumferential seal in the aspiration body 182 so that the plunger head 596 and the aspiration body 182 collectively define an aspiration chamber 598. The aspiration body 182 includes a distal aspiration end 5100 to which the second tube end 162 is (removably or permanently) connected such that the tube lumen 166 is fluidically connected to the aspiration chamber 598. The aspiration body 182 also includes an aspiration sidewall opening 5102 at a location longitudinally between the proximal and distal aspiration ends 592, 5100. As will be discussed below, the aspiration sidewall opening 5102 selectively opens the aspiration chamber 598 to atmosphere.

Although the aspiration assembly 180 has been described as using the aspiration body 182 and the plunger 184 to generate the negative pressure, the aspiration assembly 180 could include any other device(s)/component(s) for generating the negative pressure, such as, for example, a bellows or a pump. Further, although the aspiration assembly 180 is manipulated to generate the negative pressure, the aspiration assembly 180 may also be manipulated to generate positive pressure. For example, the “positive pressure” use may be desirable if the user wanted to inject a fluid (e.g., blood and/or saline) from the aspiration chamber 598 into the patient. One of ordinary skill in the art will be readily able to provide any suitable negative or positive pressure as desired, in any manner, for a particular use environment.

As shown in FIG. 1, the device includes a fluidic channel 1104 that places the access assembly 102 in fluid connection with the aspiration assembly 180. At least the aspiration chamber 598, the tube lumen 166, and the access housing lumen 120 collectively at least partially define the fluidic channel 1104. The tube lumen 166 thus at least partially defines the fluidic channel 1104 between the access assembly 102 and the aspiration assembly 180. The needle lumen 110 may also at least partially define the fluidic chamber 1104 when the needle 104 is in the deployed position. Therefore, when the needle 104 is in the deployed position, the fluidic channel 1104 extends from the distal need tip 112, through the needle lumen 110, the access housing lumen 120, and the tube lumen 166, and to the aspiration chamber 598.

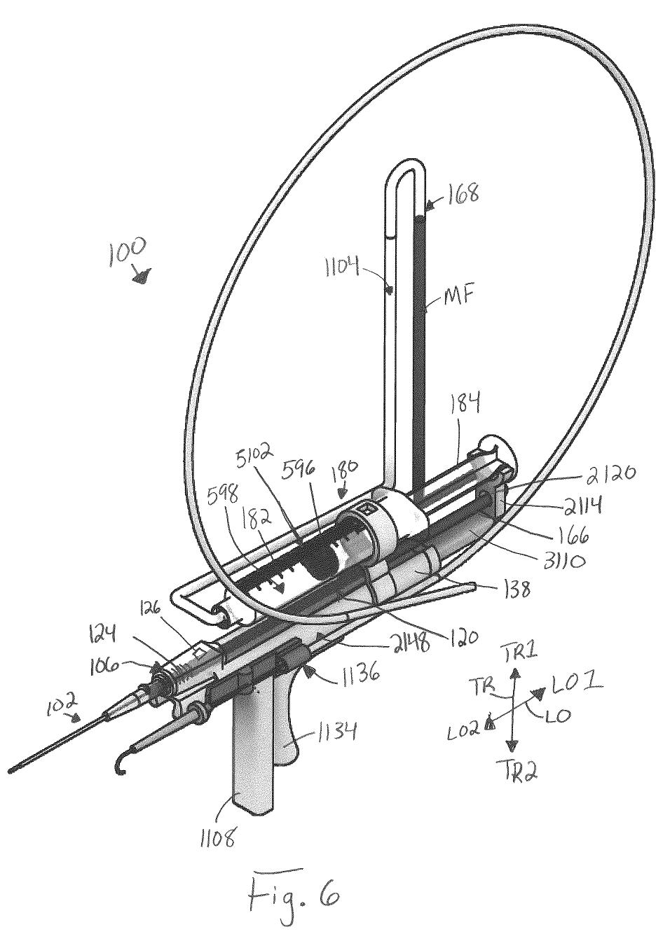

The aspiration assembly 180 is selectively operable to generate negative pressure in the fluidic channel 1104 when the device 100 is in an aspiration mode. In the aspiration mode, the plunger head 596 is in a first, closed position in which the aspiration chamber 598 is not open to atmosphere (FIG. 1). In particular, the plunger head 596, when in the aspiration mode, is located longitudinally distal of the aspiration sidewall opening 5102 so that the aspiration chamber 598 is not fluidically connected to the aspiration sidewall opening 5102 and, thus, is not open to atmosphere. As shown in FIGS. 1 and 6, the plunger 184 may be moved longitudinally in a proximal direction LO1, which responsively causes the plunger head 596 to move in and relative to the aspiration body 182. Because the device 100 is in the aspiration mode, the longitudinal movement of the plunger head 596 generates the negative pressure in the fluidic channel 1104, which may be used to draw fluid into the fluidic channel 1104. Fluid being drawn into the manometer tube portion 168 via the negative pressure results in the presence of manometer fluid (shown in the Figures as “MF”) in the manometer tube portion 168.

The plunger head 596 may be moved longitudinally proximally to generate the negative pressure until at least a portion of the plunger head 596 reaches a second, open position in which the aspiration chamber 598 is at least partially open to atmosphere (FIG. 6). In particular, the plunger head 596, when in the open position, is located longitudinally proximally beyond at least a portion of the aspiration sidewall opening 5102 so that the aspiration chamber 598 is fluidically connected to the aspiration sidewall opening 5102 and thus is open to atmosphere. The opening of the aspiration chamber 598 to atmosphere responsively transitions the device 100 from the aspiration mode to the manometer mode. In the manometer mode, the manometer fluid MF (i.e., the fluid in the manometer tube portion 168) is visually accessible for pressure measuring. The visual access can be any user-perceptible access.

One or more portions of the tube body 164 (e.g., the manometer tube portion 168) or the entirety of the fluid tube 158 may be at least partially translucent or transparent to facilitate visual observation of fluid in the tube lumen 166. Furthermore, at least one of the aspiration body 182 and the access housing 114 (e.g., the access housing head portion 116 and/or the access housing body portion 188) may be fully or partially translucent or transparent for visual observation of fluid in at least one of the aspiration chamber 598 and the access housing lumen 120.

As shown in FIGS. 1-3, the device 100 may include a trigger 1106 operatively connected to the plunger 184 of the aspiration assembly 180 such that selective manipulation of the trigger 1106 moves the plunger 184 (and, accordingly, the plunger head 596) relative to the aspiration body 182. The trigger 1106 includes a transversely extending trigger gripping portion 1108 and a longitudinally extending trigger body portion 3110. The trigger 1106 is connected and longitudinally slidable relative to the barrel 138. In particular, the trigger body portion 3110 is slidably received in a guiding groove 3112 of the barrel 138. The guiding groove 3112 is at least partially formed in the second barrel surface 242 and extends longitudinally along the barrel 138. A user's selective manipulation of the trigger 1106 (e.g., at the trigger gripping portion 1108) responsively slides the trigger body portion 3110 longitudinally along the guiding groove 3112.

The trigger body portion 3110 may be connected to the plunger 184 via a trigger connector 2114. As shown in FIG. 2, the trigger connector 2114 has a trigger connector body 2116 connected (removably or permanently) to the trigger body portion 3110 such that the trigger connector 2114 is linked for movement with the trigger 1106. The trigger connector body 2116 includes a trigger opening 2118 for receiving a portion of the trigger body portion 3110. When received in the trigger opening 2118, the trigger body portion 3110 may be retained in the trigger opening 2118 and connected to the trigger connector body 2116 via an interference or frictional fit between the trigger body portion 3110 and the portions of the trigger connector body 2116 that define the trigger opening 2118. The trigger connector 2114 and the trigger body portion 3110, however, may be connected or retained relative to one another in any desirable manner. Furthermore, the trigger connector 2114 may be formed integrally with the trigger 1106 as a one-piece, unitary component, or separately from the trigger 1106 and subsequently permanently or removably connected to the trigger 1106.

The trigger connector body may also include a tube opening 2120 through which a portion of the tube body 164 extends. As shown in FIGS. 1 and 6, via the tube opening 2120, the trigger connector 2114 slides longitudinally along a portion of the tube body 164 during a longitudinal movement of the trigger 1106.

As shown in FIGS. 1-2, the trigger connector 2114 includes a plunger engagement portion 2122 that engages a portion of the plunger 184 and thus connects the plunger 184 to the trigger body portion 3110. The plunger engagement portion 2122 is defined by first and second connector arms 2124, 2126 that extend from the trigger connector body 2116 and collectively form a curved, rounded, or arcuate plunger engagement surface 2128 on which a portion of the plunger 184 selectively rests, though the plunger engagement surface 2128 may have any other desired form. A longitudinal trigger connector end face 2130 at the plunger engagement portion 2122 and/or the trigger connector body 2116 selectively engages the plunger flange 1132 of the plunger 184. Through this engagement, the trigger connector 2114 urges the plunger 184 in the longitudinal proximal direction LO1 in response to the trigger 1106 being moved in the longitudinal proximal direction LO1.

As shown in FIGS. 1-3, a hand rest 1134 is attached to the second barrel surface 242. The hand rest 1134 may be formed integrally with the barrel 138 as a one-piece, unitary component, or separately from the barrel 138 and subsequently permanently or removably connected to the barrel 138. In either case, the hand rest 1134 and the barrel 138 may be configured such that the hand rest 1134 is not moveable relative to the barrel 138 during use of the device 100.

As shown in FIG. 1, the device 100 may include a guidewire introducer assembly 1136 that includes a guidewire housing 1138, a guidewire introducer assembly connector 1140, and a guidewire introducer head 1142. At least a proximal end 1144 of a guidewire 1146 is disposed within the guidewire housing 1138, and the guidewire housing 1138 is connected to the first barrel surface 240 and/or one of first and second barrel sidewalls 2148, 2150 of the barrel 138 via the guidewire introducer assembly connector 1140. As shown in FIG. 2, the first and second barrel sidewalls 2148, 2150 may extend transversely between the first and second barrel surfaces 240, 242. At least one of the first and second barrel sidewalls 2148, 2150, along certain longitudinal portions of the barrel 138, may form a continuous surface with the first barrel surface 240. In the configuration shown in FIG. 1, the guidewire housing 1138 is removably connected to the first barrel sidewall 2148 via the guidewire introducer assembly connector 1140.

The guidewire housing 1138 may be rigid or flexible, and is of sufficient length to hold a predetermined length of the guidewire 1146. The guidewire introducer assembly connector 1140 is configured to allow the movement of the guidewire 1146 from a distal end 1139 of guidewire housing 1138 along a guidewire feed region 1152 of the guidewire introducer assembly connector 1140 and into an introducer head lumen 1154 of the guidewire introducer head 1142. As shown in FIG. 1, a distal end 1156 of the guidewire 1146 may extend from the introducer head lumen 1154 prior to use of the guidewire 1146. Further, the guidewire introducer head 1142 (and, thus, the introducer head lumen 1154) is separate from the access assembly 102 (and, thus, the access housing lumen 120) so that the guidewire 1146 does not pass through the access housing 114 and/or the needle 104 as the guidewire 1146 is deployed from the device 100.

It is contemplated that at least a portion of the guidewire introducer assembly 1136 may be (directly or indirectly) connected to at least a portion of the catheter 128 so that when the catheter 128 is deployed into the vessel and the needle 104 is retracted, the guidewire 1146 may be passed forward directly down the catheter lumen 134 without having to first move the device 100 into a position in which the introducer head lumen 1154 is aligned with the catheter lumen 134. For example, the guidewire introducer head 1142 or another portion of the guidewire introducer assembly 1136 may be (directly or indirectly) connected to the catheter 128 so that the introducer head lumen 1154 is fluidically connected to the catheter lumen 134. The introducer head lumen 1154 may also or instead be fluidically connected to the catheter lumen 134 through the catheter projection 136.

The guidewire feed region 1152 may include a protrusion 1158 between the distal end 1139 of the guidewire housing 1138 and the guidewire introducer head 1142, over which guidewire 1146 passes. The guidewire feed region 1152 is generally configured to be accessible by the thumb of the user while gripping the hand rest 1134 and the trigger gripping portion 1108 with one hand; however, the device 100 may be configured such that the guidewire feed region 1152 is accessible by a finger of the one hand of the user gripping the hand rest 1134 and the trigger gripping portion 1108. The thumb of the user can be used to direct the guidewire 1146 longitudinally distally through the guidewire introducer head 1142. Of course, the thumb of the user may also be used to retract the guidewire 1142 in the opposite direction if desired. The protrusion 1158 may serve to provide tactile feedback to the user while advancing (or retracting) the guidewire 1146, as well as to provide a raised surface to press against while directing the guidewire 1146 to assist in the gripping of the guidewire 1146 to increase the efficiency of movement of the guidewire 1146. The protrusion 1158, however, can be replaced with other suitable structures configured to assist a user in moving the guidewire 1146 along the guidewire feed region 1152 in either longitudinal direction.

The barrel sidewall 2148, 2150 to which the guidewire introducer assembly 1136 is joined to may be selected based on the user's preferred hand. For example, the guidewire introducer assembly 1136 in FIG. 1 is configured for use with a user's right hand and, thus, the guidewire introducer assembly 1136 is connected to the first barrel sidewall 2148 (i.e., connected to the “left” side of the device 100 in the orientation of FIG. 1). The device 100 may be configured for use with a user's left hand. In the “left-hand” configuration, the guidewire introducer assembly 1136 may be connected to the second barrel sidewall 2150 (i.e., to the “right” side of the device 100 in the orientation of FIG. 1). The guidewire introducer assembly 1136 may be configured to be removably connected to the barrel 138 via the guidewire introducer assembly connector 1140 such that the guidewire introducer assembly 1136 can be switched between the first and second barrel sidewalls 2148, 2150 as desired.

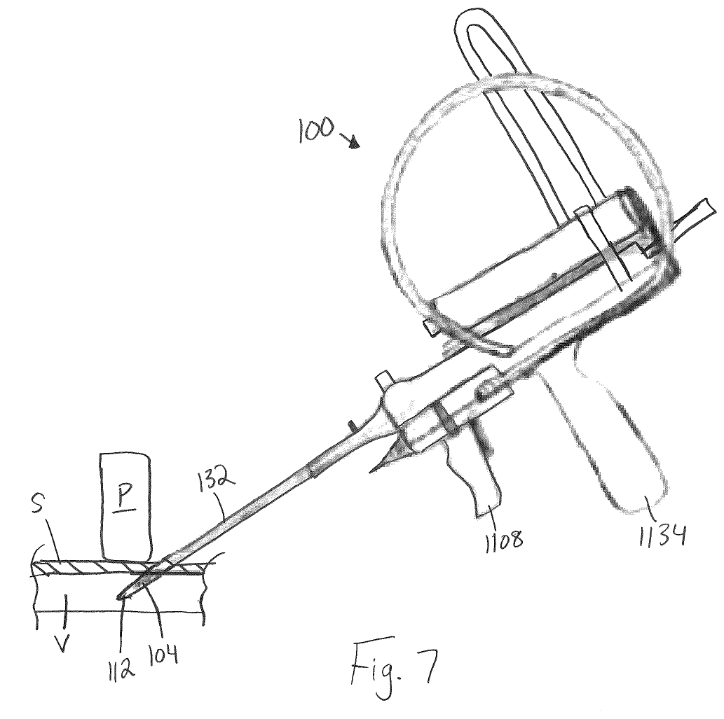

FIGS. 7-16 depict an example sequence of operating one configuration of the device 100. The device 100 is configured for one-handed operation by a user. Therefore, the below text describes the device 100 being held and operated with a “procedural hand” of the user. The user's procedural hand may be the user's dominant hand and/or the user's preferred hand for holding and operating the device 100. Furthermore, although the device 100 is configured for one-handed operation, this does not preclude the user from using the device with more than one hand if they so desire.

As shown in FIG. 7, the device 100 as shown in FIG. 1 is provided. Prior to use, the device 100 may be manipulated such that the device 100 is in the aspiration mode and the needle 104 is in the deployed position. The device 100 may be gripped with the user's procedural hand such that a portion of the user's procedural hand rests on the hand rest 1134. At least the distal needle tip 112 is inserted through a patient's skin S into a vasculature V of a patient. A portion of the catheter body 132 may also be inserted into the patient's skin S during the insertion of the distal needle tip 112. The user may operate an ultrasound probe P with their other, non-procedural hand to help guide the insertion of the needle 104 into the vasculature V.

As shown in FIG. 8, with the distal needle tip 112 inserted, the user may use at least one of the digits on their procedural hand to operate the aspiration assembly 180 to generate negative pressure in the fluidic channel 1104. In particular, the user may urge the trigger gripping portion 1108 longitudinally proximally toward the hand rest 1134. The trigger body portion 3110 responsively longitudinally slides along the guiding groove 3112 of the barrel 138, which responsively urges the plunger 184 and the plunger head 596 in the longitudinal proximal direction LO1 toward the aspiration sidewall opening 5102. Because the device 100 is in the aspiration mode, the plunger head 596 generates the negative pressure in the aspiration chamber 598 and the remainder of the fluidic chamber 1104 as the plunger 184 moves longitudinally. Via the negative pressure, fluid (here, blood B) from the patient is drawn through the distal needle tip 112, through the fluidic channel 1104, and into manometer tube portion 168 (as shown here, the first manometer tube leg 474).

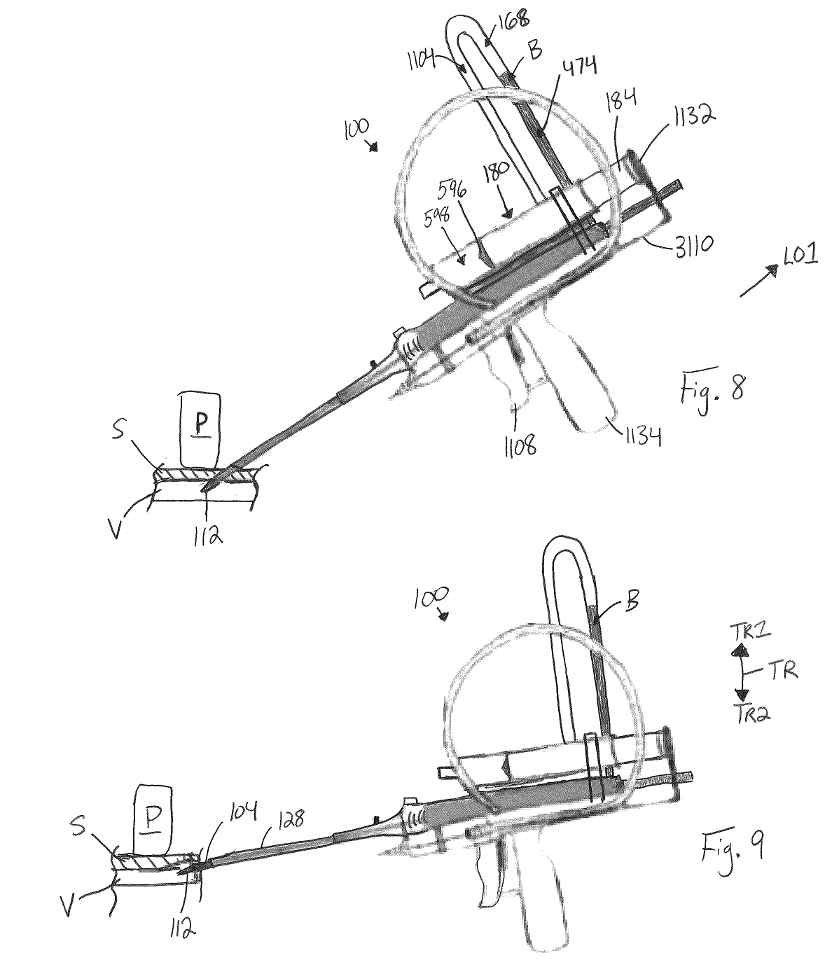

As shown in FIG. 9 the device 100 may be pivoted transversely (such as in the second transverse direction TR2), if desired, once the aspirated blood B is visible so that the needle 104 (and, optionally, the catheter 128) is more longitudinally aligned with the vasculature V than when the needle 104 was first introduced into the vasculature V. As shown in FIG. 10, once the device 100 is in the desired alignment, the user may continue to urge the trigger 1106 longitudinally proximally LO1 with at least one of their procedural hand digits to move the plunger 184 and aspirate more blood B into the first manometer tube leg 474, which increases the amount of blood B and the transverse level of blood B in the first manometer tube leg 474.

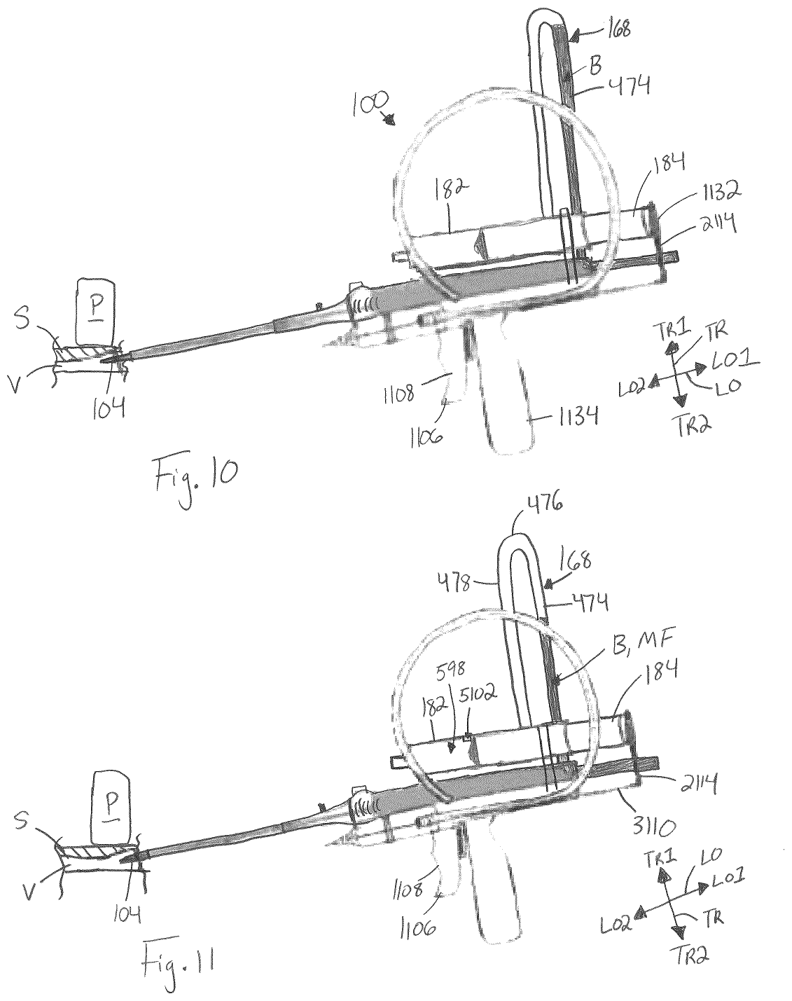

As shown in FIG. 11, the user continues to “squeeze” the trigger 1106 to transition the device 100 from the aspiration mode to the manometer mode. In particular, the plunger 184, via the trigger 1106, is moved longitudinally proximally beyond at least a portion of the aspiration sidewall opening 5102 to open the aspiration chamber 598 to atmosphere and responsively transition the device 100 to the manometer mode. The manometer tube portion 168, now that the device 100 is in the manometer mode, forms a manometer. Because of the connection to atmosphere, a fluidic pressure in the manometer tube portion 168 normalizes to a fluidic pressure of the vasculature V after the device 100 transitions to the manometer mode.

The blood B in the first manometer tube leg forms the manometer fluid MF that can be visually monitored to responsively determine whether the vasculature V that the needle 104 is inserted into is a target vasculature within the patient. For example, the target vasculature may be a vein. When the device transitions to the manometer mode with the needle 104 inserted into the vein (as is shown in FIG. 11), the transverse level of blood B in the first manometer tube leg 474 “falls” or “decreases” in the second transverse direction TR2 as a result of the fluidic pressure in the manometer tube portion 168 normalizing to a relative low fluidic pressure of the vein. However, if the needle 104 was undesirably inserted into an artery instead of the vein, the transverse level of blood B in the first manometer tube leg 474 may “rise” or “increase” in the first transverse direction TR1 as a result of the fluidic pressure in the manometer tube portion 168 normalizing to a relative high fluidic pressure of the artery. Under certain conditions, the pressure normalizing during arterial access may responsively cause a portion of the blood B in the manometer tube portion 168 to flow past the inflection point 476 and into at least the second manometer tube leg 472 (and, possibly, all the way to the aspiration chamber 598).

Accordingly, because of the pressure differences between veins and arteries, the user may visually observe how the blood B in at least the manometer tube portion 168 reacts once the device 100 is manipulated to the manometer mode to help confirm insertion of the device 100 into the target vasculature. The ultrasound probe P may also help assist with the positioning and/or the confirmation of the needle 104 in the target vasculature.

If the user determines, based on the visual observation, that the device 100 is not inserted into the target vasculature V, the user may reposition the device 100. For example, the user may withdraw the device 100 from the patient and reinsert the device 100 in a similar manner as described above. Once the device is reinserted, the user may manipulate the device 100 as described above into a desired alignment, and to visually observe if the device 100 is inserted in the target vasculature via the manometer tube portion 168. The ultrasound probe P may once again be used to help assist with the positioning and/or the confirmation of the needle 104 in the target vasculature.

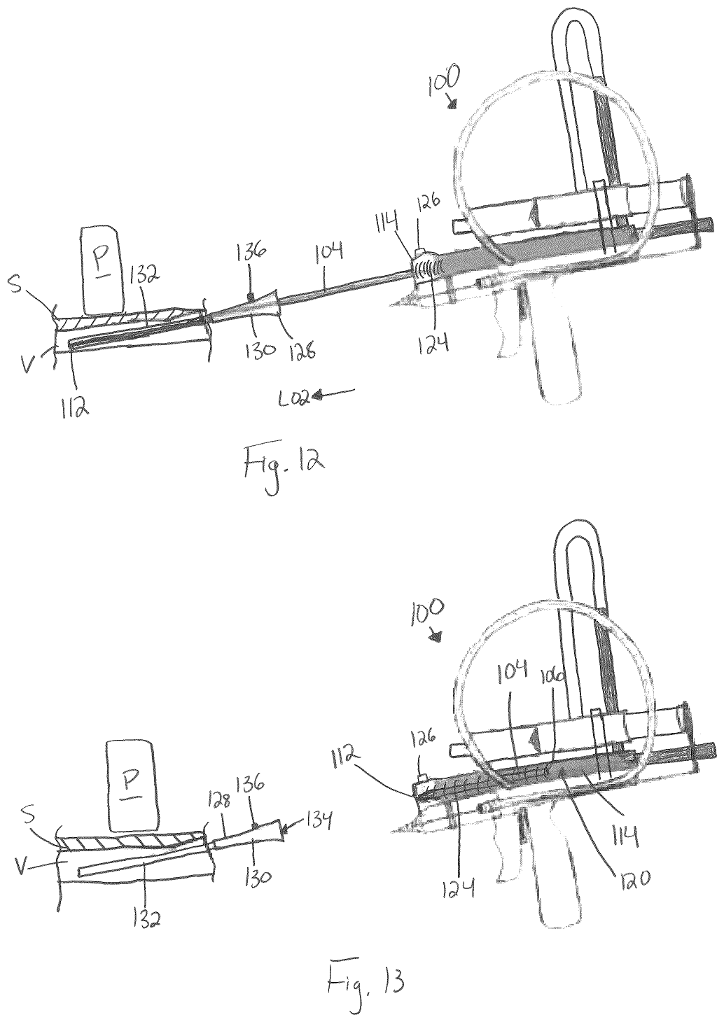

As shown in FIG. 12, if the user determines that the device 100 is inserted into the target vasculature V (here, the vein V), the user may act against the catheter projection 136 with at least one procedural hand digit to uncouple the catheter 128 from the access housing 114, and then slide the catheter 128 along the needle 104 in the longitudinally distal direction LO2 so that at least a portion of the catheter body 132 is inserted or further inserted into the vein V. As shown in FIGS. 12-13, with the catheter 128 inserted in the vein V, the user may depress the retraction trigger 126 with at least one procedural hand digit to responsively cause the spring 124 to move the needle 104 to the retracted position inside the access housing 114, while maintaining the catheter 128 inserted in the vein V.

As shown in FIG. 14, if desired, the user may position the guidewire introducer head 1142 adjacent to or inside the catheter lumen 134 at the catheter hub 130 so that the catheter lumen 134 is aligned or in fluid connection with the introducer head lumen 1154. The user may then direct the distal end 1156 of the guidewire 1146 through the catheter lumen 134 to locate at least the distal end 1156 of the guidewire 1146 in the vein V. For example, the user may use their procedural hand thumb or any other procedural hand digit to advance the guidewire 1146 longitudinally distally along the guidewire feed region 1152, which responsively directs the guidewire 1146 through the introducer head lumen 1154, into the catheter lumen 134 and into the vein V. The ultrasound probe P may be used during and throughout the insertion of the guidewire 1146 to provide ultrasonic guidance.

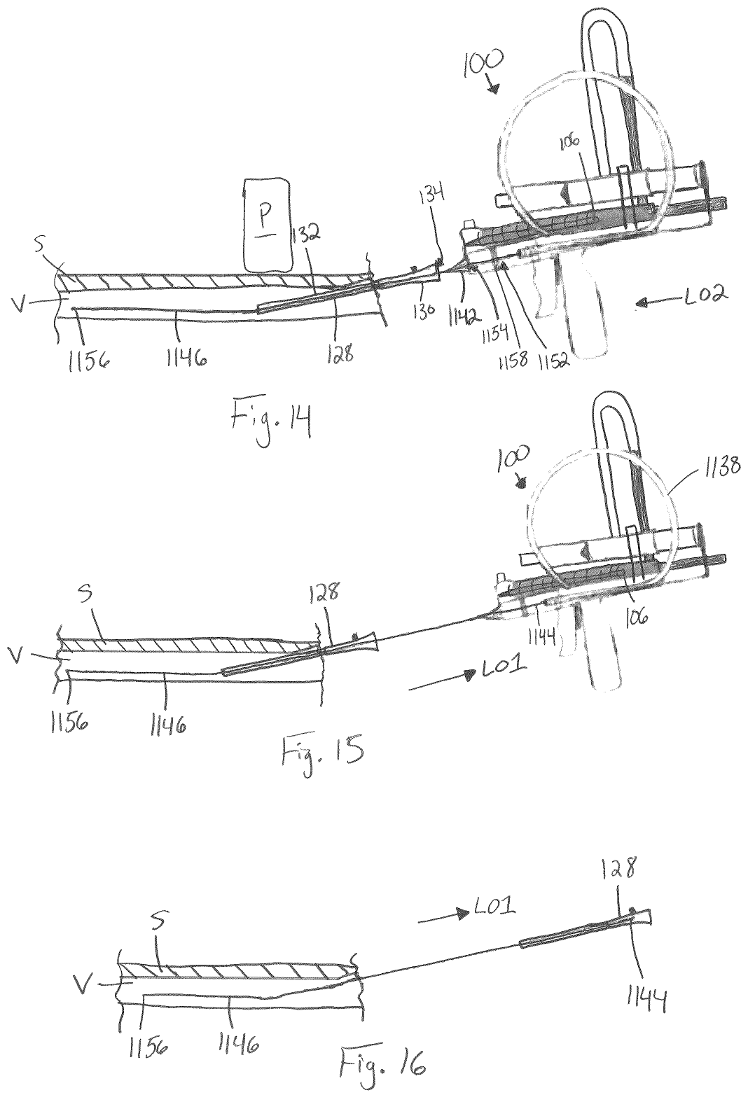

As shown in FIG. 15, the user may release the ultrasound probe P from their non-procedural hand, and then use their non-procedural hand to maintain the guidewire 1146 in place within the catheter 128 as the user uses their procedural hand to remove the device 100 from the guidewire 1146 in the longitudinally proximal direction LO1, all while the guidewire 1146 and the catheter 128 are maintained in place in the vein V. As shown in FIG. 16, the proximal end 1144 of the guidewire 1146 may be released from the guidewire housing 1138 once the device 100 is removed from the guidewire 1146. With the device 100 removed from the guidewire 1146 and the guidewire 1146 in the vein V, the user may remove the catheter 128 from the guidewire 1146 in the longitudinally proximal direction LO1, while maintaining the guidewire 1146 in the vein V. Because the guidewire 1146 is maintained in the vein V, if desired, the user may advance any other medical tools over the guidewire 1146 into the vein V.

Portions of at least the access assembly 102, the fluid tube 158, and the aspiration assembly 180 may be selectively removed from the remainder of the device 100 after use, sanitized, and replaced on the device 100 for a future use episode. Alternatively or additionally, portions of at least the access assembly 102, the fluid tube 158, and the aspiration assembly 180 may be selectively removed after use, as consumables, and replaced with corresponding structures for a future use episode.

Although the device 100 has been described as being useful with a patient in a medical setting, the device 100 may be configured to be applicable in fields outside the field of medicine. Further, although the device 100 has been described as being used to access to a patient's vasculature V, the device 100 may be used to access any other lumen in the patient.

While aspects of this disclosure have been particularly shown and described with reference to the example aspects above, it will be understood by those of ordinary skill in the art that various additional aspects may be contemplated. For example, the specific methods described above for using the apparatus are merely illustrative; one of ordinary skill in the art could readily determine any number of tools, sequences of steps, or other means/options for placing the above-described apparatus, or components thereof, into positions substantively similar to those shown and described herein. In an effort to maintain clarity in the Figures, certain ones of duplicative components shown have not been specifically numbered, but one of ordinary skill in the art will realize, based upon the components that were numbered, the element numbers which should be associated with the unnumbered components; no differentiation between similar components is intended or implied solely by the presence or absence of an element number in the Figures. Any of the described structures and components could be integrally formed as a single unitary or monolithic piece or made up of separate sub-components, with either of these formations involving any suitable stock or bespoke components and/or any suitable material or combinations of materials; however, certain of the chosen material(s) should be biocompatible for many applications. Any of the described structures and components could be disposable or reusable as desired for a particular use environment. Any component could be provided with a user-perceptible marking to indicate a material, configuration, at least one dimension, or the like pertaining to that component, the user-perceptible marking potentially aiding a user in selecting one component from an array of similar components for a particular use environment. A “predetermined” status may be determined at any time before the structures being manipulated actually reach that status, the “predetermination” being made as late as immediately before the structure achieves the predetermined status. The term “substantially” is used herein to indicate a quality that is largely, but not necessarily wholly, that which is specified—a “substantial” quality admits of the potential for some relatively minor inclusion of a non-quality item. Though certain components described herein are shown as having specific geometric shapes, all structures of this disclosure may have any suitable shapes, sizes, configurations, relative relationships, cross-sectional areas, or any other physical characteristics as desirable for a particular application. Any structures or features described with reference to one aspect or configuration could be provided, singly or in combination with other structures or features, to any other aspect or configuration, as it would be impractical to describe each of the aspects and configurations discussed herein as having all of the options discussed with respect to all of the other aspects and configurations. A device or method incorporating any of these features should be understood to fall under the scope of this disclosure as determined based upon the claims below and any equivalents thereof.

Other aspects, objects, and advantages can be obtained from a study of the drawings, the disclosure, and the appended claims.

1. An access device, comprising:
an access assembly for percutaneously accessing an anatomical space in a patient;
an aspiration assembly selectively operable to generate negative pressure in a fluidic channel of the device, the fluidic channel placing the access assembly in fluid connection with the aspiration assembly; and
a fluid tube having a first tube end connected to the access assembly, a second tube end connected to the aspiration assembly, and a tube body extending between the first and second tube ends, the tube having a tube lumen that extends between the first and second tube ends and at least partially defines the fluidic channel between the access assembly and the aspiration assembly, the tube body defining a manometer tube portion spaced apart from both the first and second tube ends, the manometer tube portion selectively acting as at least part of a manometer when the device is selectively in a manometer mode, the manometer tube portion having first and second manometer tube ends and an inflection point changing the extension direction of the manometer tube portion at a location spaced apart from both the first and second manometer tube ends.

2. The device of claim 1, further comprising a trigger operatively connected to the aspiration assembly, wherein selective manipulation of the trigger operates the aspiration assembly to generate the negative pressure.

3. The device of claim 2, wherein the aspiration assembly includes an aspiration body and a plunger having a plunger head in the aspiration body, the aspiration body and plunger head collectively defining an aspiration chamber, the aspiration chamber being fluidically connected to the tube lumen and forming a portion of the fluidic channel, the trigger being operatively connected to the plunger such that the selective manipulation of the trigger moves the plunger relative to the aspiration body to generate the negative pressure.

4. The device of claim 3, wherein the aspiration body is selectively openable to atmosphere, the device having
an aspiration mode in which the plunger head is in a first, closed position in which the aspiration chamber is not open to atmosphere, wherein selective manipulation of the trigger generates the negative pressure when the device is in the aspiration mode, and
the manometer mode in which the plunger head is in a second, open position in which the aspiration chamber is open to atmosphere and the manometer tube portion is configured to function as a manometer.

5. The device of claim 4, wherein the aspiration body includes an aspiration body sidewall opening selectively opening the aspiration chamber to atmosphere, the selective manipulation of the trigger longitudinally [moves] moving the plunger relative to the aspiration body, the device being in the aspiration mode as the plunger is moved longitudinally in a first direction toward the aspiration body sidewall opening, the device transitioning to the manometer mode responsive to movement of the plunger head longitudinally in the first direction beyond at least a portion of the aspiration body sidewall opening to open the aspiration chamber to atmosphere.

6. The device of claim 4, wherein
when the device is in the aspiration mode, the selective manipulation of the trigger to generate negative pressure draws fluid into the manometer tube portion to produce manometer fluid, and
when the device is in the manometer mode, the manometer fluid is visually accessible for pressure measuring.

7. The device of claim 2, further comprising a body to which the access assembly, the aspiration assembly, and the trigger are connected.

8-10. (canceled)

11. The device of claim 7, further comprising:
a body to which the access assembly and the aspiration assembly are connected; and
a guidewire introducer assembly, the guidewire introducer assembly including
a guidewire introducer assembly connector for connecting the guidewire introducer assembly to the body,
a guidewire housing connected to the guidewire introducer assembly connector, the guidewire housing being configured to selectively receive a proximal end of a guidewire, and
a guidewire introducer head connected to the guidewire introducer assembly connector, the guidewire introducer head being separate from the access assembly, wherein the guidewire is selectively advanced through the guidewire introducer head into the anatomical space of the patient.

12. The device of claim 11, wherein the guidewire assembly further includes a protrusion against which the guidewire is pressed concurrently with direction of the guidewire through the guidewire introducing head.

13. The device of claim 1, wherein the access assembly includes
an access housing connected to the first tube end, the access housing having an inner access housing lumen fluidically connected to the tube lumen, the inner access housing lumen forming a portion of the fluidic channel; and
a hollow needle connected to the access housing.

14. (canceled)

15. The device of claim 13, wherein the access housing includes a needle retraction mechanism for selectively moving the needle between deployed and retracted positions.

16. (canceled)

17. The device of claim 1, further comprising a guidewire introducer assembly, the guidewire introducer assembly including
a guidewire introducer assembly connector for connecting the guidewire introducer assembly to a body of the device, the access assembly and the aspiration assembly being connected to the body;
a guidewire housing connected to the guidewire introducer assembly connector, the guidewire housing being configured to selectively receive a proximal end of a guidewire; and
a guidewire introducer head connected to the guidewire assembly connector, the guidewire introducer head being separate from the access assembly, wherein the guidewire is selectively advanced through the guidewire introducer head into the anatomical space of the patient.

18. The device of claim 1, wherein the device is configured for one-handed operation by a user.

19. A method for accessing an anatomical space in a patient, the method comprising:
providing the device of claim 1;
inserting a portion of the access assembly into the patient;
operating the aspiration assembly to generate the negative pressure;
via the negative pressure, aspirating fluid from the patient into the manometer tube portion;
manipulating the device into the manometer operating mode such that the manometer tube portion acts as at least part of a manometer; and
with the device in the manometer operating mode, monitoring the fluid within the manometer tube portion to responsively determine whether the inserted portion of the access assembly is in a target anatomical space within the patient.

20. The method of claim 19, wherein the device includes a guidewire, the method further comprising:
with the inserted portion of the access assembly maintained in the target anatomical space within the patient, directing the guidewire through at least a portion of the access assembly and into the target anatomical space to locate at least a portion of the guidewire in the target anatomical space.

22. The device of claim 1, wherein the extension direction of the manometer tube portion changes by at least 45° at the inflection point.

23. The device of claim 1, wherein the extension direction of the manometer tube portion substantially reverses at the inflection point.

24. The device of claim 2, wherein the fluidic channel is selectively openable to atmosphere, the device having
an aspiration mode in which the fluidic channel is not open to atmosphere, wherein selective manipulation of the trigger generates the negative pressure when the device is in the aspiration mode, and
the manometer mode in which the fluidic channel is open to atmosphere and the manometer tube portion is configured to function as a manometer.
